SEQUENCE LISTING (182 sequences)

SEQ ID NO: 1 - protein, 8 aa, artificial sequence
  PEPTIDE: Source of material isolated peptides from phage display library (fd-tet) that bind Bacillus anthracis spores
AARQPAGM

SEQ ID NO: 2 - protein, 8 aa, artificial sequence
  PEPTIDE: Source of material isolated peptides from phage display library (fd-tet) that bind Bacillus anthracis spores
AARQPMAS

SEQ ID NO: 3 - protein, 8 aa, artificial sequence
  PEPTIDE: Source of material isolated peptides from phage display library (fd-tet) that bind Bacillus anthracis spores
AARSVTDS

SEQ ID NO: 4 - protein, 8 aa, artificial sequence
  PEPTIDE: Source of material isolated peptides from phage display library (fd-tet) that bind Bacillus anthracis spores
ADRVPYPS

SEQ ID NO: 5 - protein, 8 aa, artificial sequence
  PEPTIDE: Source of material isolated peptides from phage display library (fd-tet) that bind Bacillus anthracis spores
AGRAGGGV

SEQ ID NO: 6 - protein, 8 aa, artificial sequence
  PEPTIDE: Source of material isolated peptides from phage display library (fd-tet) that bind Bacillus anthracis spores
AGRGPGLP

SEQ ID NO: 7 - protein, 8 aa, artificial sequence
  PEPTIDE: Source of material isolated peptides from phage display library (fd-tet) that bind Bacillus anthracis spores
AHREMPQG

SEQ ID NO: 8 - protein, 8 aa, artificial sequence
  PEPTIDE: Source of material isolated peptides from phage display library (fd-tet) that bind Bacillus anthracis spores
ANRVPPTS

SEQ ID NO: 9 - protein, 8 aa, artificial sequence
  PEPTIDE: Source of material isolated peptides from phage display library (fd-tet) that bind Bacillus anthracis spores
ARPSDGLS

SEQ ID NO: 10 - protein, 8 aa, artificial sequence
  PEPTIDE: Source of material isolated peptides from phage display library (fd-tet) that bind Bacillus anthracis spores
ARSAGPLP

SEQ ID NO: 11 - protein, 8 aa, artificial sequence
  PEPTIDE: Source of material isolated peptides from phage display library (fd-tet) that bind Bacillus anthracis spores
ARSALPSS

SEQ ID NO: 12 - protein, 8 aa, artificial sequence
  PEPTIDE: Source of material isolated peptides from phage display library (fd-tet) that bind Bacillus anthracis spores
ARSNPALT

SEQ ID NO: 13 - protein, 8 aa, artificial sequence
  PEPTIDE: Source of material isolated peptides from phage display library (fd-tet) that bind Bacillus anthracis spores
ARSQPALS

SEQ ID NO: 14 - protein, 8 aa, artificial sequence
  PEPTIDE: Source of material isolated peptides from phage display library (fd-tet) that bind Bacillus anthracis spores
ARSSPSLA

SEQ ID NO: 15 - protein, 8 aa, artificial sequence
  PEPTIDE: Source of material isolated peptides from phage display library (fd-tet) that bind Bacillus anthracis spores
ASRDGAVM

SEQ ID NO: 16 - protein, 8 aa, artificial sequence
  PEPTIDE: Source of material isolated peptides from phage display library (fd-tet) that bind Bacillus anthracis spores
ASRSSGAL

SEQ ID NO: 17 - protein, 8 aa, artificial sequence
  PEPTIDE: Source of material isolated peptides from phage display library (fd-tet) that bind Bacillus anthracis spores
ASRTSGLP

SEQ ID NO: 18 - protein, 8 aa, artificial sequence
  PEPTIDE: Source of material isolated peptides from phage display library (fd-tet) that bind Bacillus anthracis spores
ATRPASSM

SEQ ID NO: 19 - protein, 8 aa, artificial sequence
  PEPTIDE: Source of material isolated peptides from phage display library (fd-tet) that bind Bacillus anthracis spores
ATRTAPTS

SEQ ID NO: 20 - protein, 8 aa, artificial sequence
  PEPTIDE: Source of material isolated peptides from phage display library (fd-tet) that bind Bacillus anthracis spores
ATRTTSPM

SEQ ID NO: 21 - protein, 8 aa, artificial sequence
  PEPTIDE: Source of material isolated peptides from phage display library (fd-tet) that bind Bacillus anthracis spores
AVRDQPNL

SEQ ID NO: 22 - protein, 8 aa, artificial sequence
  PEPTIDE: Source of material isolated peptides from phage display library (fd-tet) that bind Bacillus anthracis spores
DARGTTHM

SEQ ID NO: 23 - protein, 8 aa, artificial sequence
  PEPTIDE: Source of material isolated peptides from phage display library (fd-tet) that bind Bacillus anthracis spores
DDAGRGTP

SEQ ID NO: 24 - protein, 8 aa, artificial sequence
  PEPTIDE: Source of material isolated peptides from phage display library (fd-tet) that bind Bacillus anthracis spores
DKLSSSGT

SEQ ID NO: 25 - protein, 8 aa, artificial sequence
  PEPTIDE: Source of material isolated peptides from phage display library (fd-tet) that bind Bacillus anthracis spores
DLGDRSQG

SEQ ID NO: 26 - protein, 8 aa, artificial sequence
  PEPTIDE: Source of material isolated peptides from phage display library (fd-tet) that bind Bacillus anthracis spores
DPRAAVTA

SEQ ID NO: 27 - protein, 8 aa, artificial sequence
  PEPTIDE: Source of material isolated peptides from phage display library (fd-tet) that bind Bacillus anthracis spores
DPSAYSRA

SEQ ID NO: 28 - protein, 8 aa, artificial sequence
  PEPTIDE: Source of material isolated peptides from phage display library (fd-tet) that bind Bacillus anthracis spores
DRTGATLT

SEQ ID NO: 29 - protein, 8 aa, artificial sequence
  PEPTIDE: Source of material isolated peptides from phage display library (fd-tet) that bind Bacillus anthracis spores
DSRALIAP

SEQ ID NO: 30 - protein, 8 aa, artificial sequence
  PEPTIDE: Source of material isolated peptides from phage display library (fd-tet) that bind Bacillus anthracis spores
DTRETGSS

SEQ ID NO: 31 - protein, 8 aa, artificial sequence
  PEPTIDE: Source of material isolated peptides from phage display library (fd-tet) that bind Bacillus anthracis spores
DYDAVMRT

SEQ ID NO: 32 - protein, 8 aa, artificial sequence
  PEPTIDE: Source of material isolated peptides from phage display library (fd-tet) that bind Bacillus anthracis spores
DYPTKTNS

SEQ ID NO: 33 - protein, 8 aa, artificial sequence
  PEPTIDE: Source of material isolated peptides from phage display library (fd-tet) that bind Bacillus anthracis spores
EDGRGGTA

SEQ ID NO: 34 - protein, 8 aa, artificial sequence
  PEPTIDE: Source of material isolated peptides from phage display library (fd-tet) that bind Bacillus anthracis spores
EDRVFPST

SEQ ID NO: 35 - protein, 8 aa, artificial sequence
  PEPTIDE: Source of material isolated peptides from phage display library (fd-tet) that bind Bacillus anthracis spores
EERQNPSG

SEQ ID NO: 36 - protein, 8 aa, artificial sequence
  PEPTIDE: Source of material isolated peptides from phage display library (fd-tet) that bind Bacillus anthracis spores
EFTARPSS

SEQ ID NO: 37 - protein, 8 aa, artificial sequence
  PEPTIDE: Source of material isolated peptides from phage display library (fd-tet) that bind Bacillus anthracis spores
EKTPVTAT

SEQ ID NO: 38 - protein, 8 aa, artificial sequence
  PEPTIDE: Source of material isolated peptides from phage display library (fd-tet) that bind Bacillus anthracis spores
ENSRSTQM

SEQ ID NO: 39 - protein, 8 aa, artificial sequence
  PEPTIDE: Source of material isolated peptides from phage display library (fd-tet) that bind Bacillus anthracis spores
EPHPKTST

SEQ ID NO: 40 - protein, 8 aa, artificial sequence
  PEPTIDE: Source of material isolated peptides from phage display library (fd-tet) that bind Bacillus anthracis spores
EPKMQAAQ

SEQ ID NO: 41 - protein, 8 aa, artificial sequence
  PEPTIDE: Source of material isolated peptides from phage display library (fd-tet) that bind Bacillus anthracis spores
EPKPHTFS

SEQ ID NO: 42 - protein, 8 aa, artificial sequence
  PEPTIDE: Source of material isolated peptides from phage display library (fd-tet) that bind Bacillus anthracis spores
EPMRDMAS

SEQ ID NO: 43 - protein, 8 aa, artificial sequence
  PEPTIDE: Source of material isolated peptides from phage display library (fd-tet) that bind Bacillus anthracis spores
EPRAPASL

SEQ ID NO: 44 - protein, 8 aa, artificial sequence
  PEPTIDE: Source of material isolated peptides from phage display library (fd-tet) that bind Bacillus anthracis spores
EPRLAGDQ

SEQ ID NO: 45 - protein, 8 aa, artificial sequence
  PEPTIDE: Source of material isolated peptides from phage display library (fd-tet) that bind Bacillus anthracis spores
EPRLSPHS

SEQ ID NO: 46 - protein, 8 aa, artificial sequence
  PEPTIDE: Source of material isolated peptides from phage display library (fd-tet) that bind Bacillus anthracis spores
EPSRGPGS

SEQ ID NO: 47 - protein, 8 aa, artificial sequence
  PEPTIDE: Source of material isolated peptides from phage display library (fd-tet) that bind Bacillus anthracis spores
GRAATDQT

SEQ ID NO: 48 - protein, 8 aa, artificial sequence
  PEPTIDE: Source of material isolated peptides from phage display library (fd-tet) that bind Bacillus anthracis spores
ERTVATTQ

SEQ ID NO: 49 - protein, 8 aa, artificial sequence
  PEPTIDE: Source of material isolated peptides from phage display library (fd-tet) that bind Bacillus anthracis spores
ESRGVGTE

SEQ ID NO: 50 - protein, 8 aa, artificial sequence
  PEPTIDE: Source of material isolated peptides from phage display library (fd-tet) that bind Bacillus anthracis spores
ETRVPHGA

SEQ ID NO: 51 - protein, 8 aa, artificial sequence
  PEPTIDE: Source of material isolated peptides from phage display library (fd-tet) that bind Bacillus anthracis spores
EYERASQG

SEQ ID NO: 52 - protein, 8 aa, artificial sequence
  PEPTIDE: Source of material isolated peptides from phage display library (fd-tet) that bind Bacillus anthracis spores
GLRTTPNT

SEQ ID NO: 53 - protein, 8 aa, artificial sequence
  PEPTIDE: Source of material isolated peptides from phage display library (fd-tet) that bind Bacillus anthracis spores
GPESQTAS

SEQ ID NO: 54 - protein, 8 aa, artificial sequence
  PEPTIDE: Source of material isolated peptides from phage display library (fd-tet) that bind Bacillus anthracis spores
GQRTPPTT

SEQ ID NO: 55 - protein, 8 aa, artificial sequence
  PEPTIDE: Source of material isolated peptides from phage display library (fd-tet) that bind Bacillus anthracis spores
PARDPVNM

SEQ ID NO: 56 - protein, 8 aa, artificial sequence
  PEPTIDE: Source of material isolated peptides from phage display library (fd-tet) that bind Bacillus anthracis spores
VARSTGDS

SEQ ID NO: 57 - protein, 8 aa, artificial sequence
  PEPTIDE: Source of material isolated peptides from phage display library (fd-tet) that bind Bacillus anthracis spores
VDRGSATS

SEQ ID NO: 58 - protein, 8 aa, artificial sequence
  PEPTIDE: Source of material isolated peptides from phage display library (fd-tet) that bind Bacillus anthracis spores
VDRGTTLS

SEQ ID NO: 59 - protein, 8 aa, artificial sequence
  PEPTIDE: Source of material isolated peptides from phage display library (fd-tet) that bind Bacillus anthracis spores
VDRSSTTT

SEQ ID NO: 60 - protein, 8 aa, artificial sequence
  PEPTIDE: Source of material isolated peptides from phage display library (fd-tet) that bind Bacillus anthracis spores
VDRTPPSQ

SEQ ID NO: 61 - protein, 8 aa, artificial sequence
  PEPTIDE: Source of material isolated peptides from phage display library (fd-tet) that bind Bacillus anthracis spores
VDRTSSPA

SEQ ID NO: 62 - protein, 8 aa, artificial sequence
  PEPTIDE: Source of material isolated peptides from phage display library (fd-tet) that bind Bacillus anthracis spores
VGRANPSS

SEQ ID NO: 63 - protein, 8 aa, artificial sequence
  PEPTIDE: Source of material isolated peptides from phage display library (fd-tet) that bind Bacillus anthracis spores
VGREGGAV

SEQ ID NO: 64 - protein, 8 aa, artificial sequence
  PEPTIDE: Source of material isolated peptides from phage display library (fd-tet) that bind Bacillus anthracis spores
VNQTAQPA

SEQ ID NO: 65 - protein, 8 aa, artificial sequence
  PEPTIDE: Source of material isolated peptides from phage display library (fd-tet) that bind Bacillus anthracis spores
VPPPSSST

SEQ ID NO: 66 - protein, 8 aa, artificial sequence
  PEPTIDE: Source of material isolated peptides from phage display library (fd-tet) that bind Bacillus anthracis spores
VPRPDATS

SEQ ID NO: 67 - protein, 8 aa, artificial sequence
  PEPTIDE: Source of material isolated peptides from phage display library (fd-tet) that bind Bacillus anthracis spores
VPTRTPQG

SEQ ID NO: 68 - protein, 8 aa, artificial sequence
  PEPTIDE: Source of material isolated peptides from phage display library (fd-tet) that bind Bacillus anthracis spores
VPTSRADQ

SEQ ID NO: 69 - protein, 8 aa, artificial sequence
  PEPTIDE: Source of material isolated peptides from phage display library (fd-tet) that bind Bacillus anthracis spores
VPTTRETS

SEQ ID NO: 70 - protein, 8 aa, artificial sequence
  PEPTIDE: Source of material isolated peptides from phage display library (fd-tet) that bind Bacillus anthracis spores
VQPTAAPP

SEQ ID NO: 71 - protein, 8 aa, artificial sequence
  PEPTIDE: Source of material isolated peptides from phage display library (fd-tet) that bind Bacillus anthracis spores
VRPTPTDT

SEQ ID NO: 72 - protein, 8 aa, artificial sequence
  PEPTIDE: Source of material isolated peptides from phage display library (fd-tet) that bind Bacillus anthracis spores
VSDRGTAT

SEQ ID NO: 73 - protein, 8 aa, artificial sequence
  PEPTIDE: Source of material isolated peptides from phage display library (fd-tet) that bind Bacillus anthracis spores
VSDRVSPA

SEQ ID NO: 74 - protein, 8 aa, artificial sequence
  PEPTIDE: Source of material isolated peptides from phage display library (fd-tet) that bind Bacillus anthracis spores
VSPTQQQT

SEQ ID NO: 75 - protein, 8 aa, artificial sequence
  PEPTIDE: Source of material isolated peptides from phage display library (fd-tet) that bind Bacillus anthracis spores
VSQPASPS

SEQ ID NO: 76 - protein, 8 aa, artificial sequence
  PEPTIDE: Source of material isolated peptides from phage display library (fd-tet) that bind Bacillus anthracis spores
VSREAAAS

SEQ ID NO: 77 - protein, 8 aa, artificial sequence
  PEPTIDE: Source of material isolated peptides from phage display library (fd-tet) that bind Bacillus anthracis spores
VSRIPSET

SEQ ID NO: 78 - protein, 8 aa, artificial sequence
  PEPTIDE: Source of material isolated peptides from phage display library (fd-tet) that bind Bacillus anthracis spores
VSRMESTP

SEQ ID NO: 79 - protein, 8 aa, artificial sequence
  PEPTIDE: Source of material isolated peptides from phage display library (fd-tet) that bind Bacillus anthracis spores
VSRTQEHV

SEQ ID NO: 80 - protein, 8 aa, artificial sequence
  PEPTIDE: Source of material isolated peptides from phage display library (fd-tet) that bind Bacillus anthracis spores
VSTRPTET

SEQ ID NO: 81 - protein, 8 aa, artificial sequence
  PEPTIDE: Source of material isolated peptides from phage display library (fd-tet) that bind Bacillus anthracis spores
VTDARTPA

SEQ ID NO: 82 - protein, 8 aa, artificial sequence
  PEPTIDE: Source of material isolated peptides from phage display library (fd-tet) that bind Bacillus anthracis spores
VTNANSPS

SEQ ID NO: 83 - protein, 8 aa, artificial sequence
  PEPTIDE: Source of material isolated peptides from phage display library (fd-tet) that bind Bacillus anthracis spores
VTPRADST

SEQ ID NO: 84 - protein, 8 aa, artificial sequence
  PEPTIDE: Source of material isolated peptides from phage display library (fd-tet) that bind Bacillus anthracis spores
VTRDLSSS

SEQ ID NO: 85 - protein, 8 aa, artificial sequence
  PEPTIDE: Source of material isolated peptides from phage display library (fd-tet) that bind Bacillus anthracis spores
VTRGSMNT

SEQ ID NO: 86 - protein, 8 aa, artificial sequence
  PEPTIDE: Source of material isolated peptides from phage display library (fd-tet) that bind Bacillus anthracis spores
VTRNPAAS

SEQ ID NO: 87 - protein, 8 aa, artificial sequence
  PEPTIDE: Source of material isolated peptides from phage display library (fd-tet) that bind Bacillus anthracis spores
VTRNPSDA

SEQ ID NO: 88 - protein, 8 aa, artificial sequence
  PEPTIDE: Source of material isolated peptides from phage display library (fd-tet) that bind Bacillus anthracis spores
VTRNTSAS

SEQ ID NO: 89 - protein, 8 aa, artificial sequence
  PEPTIDE: Source of material isolated peptides from phage display library (fd-tet) that bind Bacillus anthracis spores
VTSASSSQ

SEQ ID NO: 90 - protein, 8 aa, artificial sequence
  PEPTIDE: Source of material isolated peptides from phage display library (fd-tet) that bind Bacillus anthracis spores
VVREPTHS

SEQ ID NO: 91 - protein, 8 aa, artificial sequence
  PEPTIDE: Source of material isolated peptides from phage display library (fd-tet) that bind Bacillus anthracis spores
VVRGSDGA

SEQ ID NO: 92 - DNA, 24 bp, ARTIFICIAL SEQUENCE
  MAT_PEPTIDE: Source of material isolated peptides from phage display library (fd-tet) that bind Bacillus anthracis spores
gcggctcgtcagcctgcggggatg

SEQ ID NO: 93 - DNA, 24 bp, ARTIFICIAL SEQUENCE
  MAT_PEPTIDE: Source of material Sequenced peptides isolated from phage display library (fd-tet) that bind Bacillus anthracis spores
gctgcgcggcagccgatggcttcg

SEQ ID NO: 94 - DNA, 24 bp, ARTIFICIAL SEQUENCE
  MAT_PEPTIDE: Source of material Sequenced peptides isolated from phage display library (fd-tet) that bind Bacillus anthracis spores
gcggctcgttctgtgactgattcg

SEQ ID NO: 95 - DNA, 24 bp, ARTIFICIAL SEQUENCE
  MAT_PEPTIDE: Source of material Sequenced peptides isolated from phage display library (fd-tet) that bind Bacillus anthracis spores
gctgatagggtgccttatccttcg

SEQ ID NO: 96 - DNA, 24 bp, ARTIFICIAL SEQUENCE
  MAT_PEPTIDE: Source of material Sequenced peptides isolated from phage display library (fd-tet) that bind Bacillus anthracis spores
gcgggtcgtgctggggggggggtg

SEQ ID NO: 97 - DNA, 24 bp, ARTIFICIAL SEQUENCE
  MAT_PEPTIDE: Source of material Sequenced peptides isolated from phage display library (fd-tet) that bind Bacillus anthracis spores
gctggtcgggggcctgggctgccg

SEQ ID NO: 98 - DNA, 24 bp, ARTIFICIAL SEQUENCE
  MAT_PEPTIDE: Source of material Sequenced peptides isolated from phage display library (fd-tet) that bind Bacillus anthracis spores
gcgcatagggagatgcctcagggg

SEQ ID NO: 99 - DNA, 24 bp, ARTIFICIAL SEQUENCE
  MAT_PEPTIDE: Source of material Sequenced peptides isolated from phage display library (fd-tet) that bind Bacillus anthracis spores
gctaatagggtgccgccgacttcg

SEQ ID NO: 100 - DNA, 24 bp, ARTIFICIAL SEQUENCE
  MAT_PEPTIDE: Source of material Sequenced peptides isolated from phage display library (fd-tet) that bind Bacillus anthracis spores
gcgaggccgtcggatggtctttcg

SEQ ID NO: 101 - DNA, 24 bp, ARTIFICIAL SEQUENCE
  MAT_PEPTIDE: Source of material Sequenced peptides isolated from phage display library (fd-tet) that bind Bacillus anthracis spores
gcgcggtcggcggggccgctgccg

SEQ ID NO: 102 - DNA, 24 bp, ARTIFICIAL SEQUENCE
  MAT_PEPTIDE: Source of material Sequenced peptides isolated from phage display library (fd-tet) that bind Bacillus anthracis spores
gctcgttcggctctgccgtcttcg

SEQ ID NO: 103 - DNA, 24 bp, ARTIFICIAL SEQUENCE
  MAT_PEPTIDE: Source of material Sequenced peptides isolated from phage display library (fd-tet) that bind Bacillus anthracis spores
gctcgttcgaatcctgcgcttacg

SEQ ID NO: 104 - DNA, 24 bp, ARTIFICIAL SEQUENCE
  MAT_PEPTIDE: Source of material Sequenced peptides isolated from phage display library (fd-tet) that bind Bacillus anthracis spores
gctcgttcgcagccggctttgtcg

SEQ ID NO: 105 - DNA, 24 bp, ARTIFICIAL SEQUENCE
  MAT_PEPTIDE: Source of material Sequenced peptides isolated from phage display library (fd-tet) that bind Bacillus anthracis spores
gcgcgttcgtcgccgagtttggcg

SEQ ID NO: 106 - DNA, 24 bp, ARTIFICIAL SEQUENCE
  MAT_PEPTIDE: Source of material Sequenced peptides isolated from phage display library (fd-tet) that bind Bacillus anthracis spores
gcttctagggatggggctgtgatg

SEQ ID NO: 107 - DNA, 24 bp, ARTIFICIAL SEQUENCE
  MAT_PEPTIDE: Source of material Sequenced peptides isolated from phage display library (fd-tet) that bind Bacillus anthracis spores
gctagtcgttctagtggtgctttg

SEQ ID NO: 108 - DNA, 24 bp, ARTIFICIAL SEQUENCE
  MAT_PEPTIDE: Source of material Sequenced peptides isolated from phage display library (fd-tet) that bind Bacillus anthracis spores
gcttcgcggacgtctggtcttccg

SEQ ID NO: 109 - DNA, 24 bp, ARTIFICIAL SEQUENCE
  MAT_PEPTIDE: Source of material Sequenced peptides isolated from phage display library (fd-tet) that bind Bacillus anthracis spores
gcgacgcgtcctgcttctagtatg

SEQ ID NO: 110 - DNA, 24 bp, ARTIFICIAL SEQUENCE
  MAT_PEPTIDE: Source of material Sequenced peptides isolated from phage display library (fd-tet) that bind Bacillus anthracis spores
gcgacgaggactgcgcctacttcg

SEQ ID NO: 111 - DNA, 24 bp, ARTIFICIAL SEQUENCE
  MAT_PEPTIDE: Source of material Sequenced peptides isolated from phage display library (fd-tet) that bind Bacillus anthracis spores
gctacgcgtacgacttctcctatg

SEQ ID NO: 112 - DNA, 24 bp, ARTIFICIAL SEQUENCE
  MAT_PEPTIDE: Source of material Sequenced peptides isolated from phage display library (fd-tet) that bind Bacillus anthracis spores
gcggtgcgggatcagccgaatctg

SEQ ID NO: 113 - DNA, 24 bp, ARTIFICIAL SEQUENCE
  MAT_PEPTIDE: Source of material Sequenced peptides isolated from phage display library (fd-tet) that bind Bacillus anthracis spores
gatgctcgggggacgacgcatatg

SEQ ID NO: 114 - DNA, 24 bp, ARTIFICIAL SEQUENCE
  MAT_PEPTIDE: Source of material Sequenced peptides isolated from phage display library (fd-tet) that bind Bacillus anthracis spores
gatgatgctggtcgggggacgccg

SEQ ID NO: 115 - DNA, 24 bp, ARTIFICIAL SEQUENCE
  MAT_PEPTIDE: Source of material Sequenced peptides isolated from phage display library (fd-tet) that bind Bacillus anthracis spores
gataagttgtcgtcttcggggacg

SEQ ID NO: 116 - DNA, 24 bp, ARTIFICIAL SEQUENCE
  MAT_PEPTIDE: Source of material Sequenced peptides isolated from phage display library (fd-tet) that bind Bacillus anthracis spores
gatcttggggatcgttctcagggg

SEQ ID NO: 117 - DNA, 24 bp, ARTIFICIAL SEQUENCE
  MAT_PEPTIDE: Source of material Sequenced peptides isolated from phage display library (fd-tet) that bind Bacillus anthracis spores
gatcctagggctgctgtgactgcg

SEQ ID NO: 118 - DNA, 24 bp, ARTIFICIAL SEQUENCE
  MAT_PEPTIDE: Source of material Sequenced peptides isolated from phage display library (fd-tet) that bind Bacillus anthracis spores
gatccgagtgcgtatagtcgggcg

SEQ ID NO: 119 - DNA, 24 bp, ARTIFICIAL SEQUENCE
  MAT_PEPTIDE: Source of material Sequenced peptides isolated from phage display library (fd-tet) that bind Bacillus anthracis spores
gatcgtactggtgctacgctgacg

SEQ ID NO: 120 - DNA, 24 bp, ARTIFICIAL SEQUENCE
  MAT_PEPTIDE: Source of material Sequenced peptides isolated from phage display library (fd-tet) that bind Bacillus anthracis spores
gattctagggcgcttattgcgccg

SEQ ID NO: 121 - DNA, 24 bp, ARTIFICIAL SEQUENCE
  MAT_PEPTIDE: Source of material Sequenced peptides isolated from phage display library (fd-tet) that bind Bacillus anthracis spores
gatactcgtgagacggggtcgtcg

SEQ ID NO: 122 - DNA, 24 bp, ARTIFICIAL SEQUENCE
  MAT_PEPTIDE: Source of material Sequenced peptides isolated from phage display library (fd-tet) that bind Bacillus anthracis spores
gattatgatgctgtgatgcggacg

SEQ ID NO: 123 - DNA, 24 bp, ARTIFICIAL SEQUENCE
  MAT_PEPTIDE: Source of material Sequenced peptides isolated from phage display library (fd-tet) that bind Bacillus anthracis spores
gattatccgactaagacgaattcg

SEQ ID NO: 124 - DNA, 24 bp, ARTIFICIAL SEQUENCE
  MAT_PEPTIDE: Source of material Sequenced peptides isolated from phage display library (fd-tet) that bind Bacillus anthracis spores
gaggatggtcgtggtggtactgcg

SEQ ID NO: 125 - DNA, 24 bp, ARTIFICIAL SEQUENCE
  MAT_PEPTIDE: Source of material Sequenced peptides isolated from phage display library (fd-tet) that bind Bacillus anthracis spores
gaggatcgggtttttccttcgacg

SEQ ID NO: 126 - DNA, 24 bp, ARTIFICIAL SEQUENCE
  MAT_PEPTIDE: Source of material Sequenced peptides isolated from phage display library (fd-tet) that bind Bacillus anthracis spores
gaggagcggcagaatccgtcgggg

SEQ ID NO: 127 - DNA, 24 bp, ARTIFICIAL SEQUENCE
  MAT_PEPTIDE: Source of material Sequenced peptides isolated from phage display library (fd-tet) that bind Bacillus anthracis spores
gagtttacggcgcggccgtcgtcg

SEQ ID NO: 128 - DNA, 24 bp, ARTIFICIAL SEQUENCE
  MAT_PEPTIDE: Source of material Sequenced peptides isolated from phage display library (fd-tet) that bind Bacillus anthracis spores
gagaagacgccggttactgcgacg

SEQ ID NO: 129 - DNA, 24 bp, ARTIFICIAL SEQUENCE
  MAT_PEPTIDE: Source of material Sequenced peptides isolated from phage display library (fd-tet) that bind Bacillus anthracis spores
gagaattctcgttcgacgcagatg

SEQ ID NO: 130 - DNA, 24 bp, ARTIFICIAL SEQUENCE
  MAT_PEPTIDE: Source of material Sequenced peptides isolated from phage display library (fd-tet) that bind Bacillus anthracis spores
gagcctcatcctaagacgtcgacg

SEQ ID NO: 131 - DNA, 24 bp, ARTIFICIAL SEQUENCE
  MAT_PEPTIDE: Source of material Sequenced peptides isolated from phage display library (fd-tet) that bind Bacillus anthracis spores
gagcctaagatgcaggctgctcag

SEQ ID NO: 132 - DNA, 24 bp, ARTIFICIAL SEQUENCE
  MAT_PEPTIDE: Source of material Sequenced peptides isolated from phage display library (fd-tet) that bind Bacillus anthracis spores
gagcctaagcctcatactttttcg

SEQ ID NO: 133 - DNA, 24 bp, ARTIFICIAL SEQUENCE
  MAT_PEPTIDE: Source of material Sequenced peptides isolated from phage display library (fd-tet) that bind Bacillus anthracis spores
gagcctatgcgtgatatggcttcg

SEQ ID NO: 134 - DNA, 24 bp, ARTIFICIAL SEQUENCE
  MAT_PEPTIDE: Source of material Sequenced peptides isolated from phage display library (fd-tet) that bind Bacillus anthracis spores
gagcctcgggcgccggcgagtctg

SEQ ID NO: 135 - DNA, 24 bp, ARTIFICIAL SEQUENCE
  MAT_PEPTIDE: Source of material Sequenced peptides isolated from phage display library (fd-tet) that bind Bacillus anthracis spores
gagccacgtcttgcgggggatcag

SEQ ID NO: 136 - DNA, 24 bp, ARTIFICIAL SEQUENCE
  MAT_PEPTIDE: Source of material Sequenced peptides isolated from phage display library (fd-tet) that bind Bacillus anthracis spores
gagcctcgtctgtctcctcattcg

SEQ ID NO: 137 - DNA, 24 bp, ARTIFICIAL SEQUENCE
  MAT_PEPTIDE: Source of material Sequenced peptides isolated from phage display library (fd-tet) that bind Bacillus anthracis spores
gagccttctcggggtcctggatcg

SEQ ID NO: 138 - DNA, 24 bp, ARTIFICIAL SEQUENCE
  MAT_PEPTIDE: Source of material Sequenced peptides isolated from phage display library (fd-tet) that bind Bacillus anthracis spores
gagagggcggctactgatcagacg

SEQ ID NO: 139 - DNA, 24 bp, ARTIFICIAL SEQUENCE
  MAT_PEPTIDE: Source of material Sequenced peptides isolated from phage display library (fd-tet) that bind Bacillus anthracis spores
gagcggactgttgcgacgacgcag

SEQ ID NO: 140 - DNA, 24 bp, ARTIFICIAL SEQUENCE
  MAT_PEPTIDE: Source of material Sequenced peptides isolated from phage display library (fd-tet) that bind Bacillus anthracis spores
gagtctaggggggttggtactgag

SEQ ID NO: 141 - DNA, 24 bp, ARTIFICIAL SEQUENCE
  MAT_PEPTIDE: Source of material Sequenced peptides isolated from phage display library (fd-tet) that bind Bacillus anthracis spores
gagactagggttcctcatggtgcg

SEQ ID NO: 142 - DNA, 24 bp, ARTIFICIAL SEQUENCE
  MAT_PEPTIDE: Source of material Sequenced peptides isolated from phage display library (fd-tet) that bind Bacillus anthracis spores
gagtatgagagggctagtcagggg

SEQ ID NO: 143 - DNA, 24 bp, ARTIFICIAL SEQUENCE
  MAT_PEPTIDE: Source of material Sequenced peptides isolated from phage display library (fd-tet) that bind Bacillus anthracis spores
ggacttcgtactactcctaatacg

SEQ ID NO: 144 - DNA, 24 bp, ARTIFICIAL SEQUENCE
  MAT_PEPTIDE: Source of material Sequenced peptides isolated from phage display library (fd-tet) that bind Bacillus anthracis spores
ggtcctgagagtcagactgcttcg

SEQ ID NO: 145 - DNA, 24 bp, ARTIFICIAL SEQUENCE
  MAT_PEPTIDE: Source of material Sequenced peptides isolated from phage display library (fd-tet) that bind Bacillus anthracis spores
gggcagaggactcctccgacgacg

SEQ ID NO: 146 - DNA, 24 bp, ARTIFICIAL SEQUENCE
  MAT_PEPTIDE: Source of material Sequenced peptides isolated from phage display library (fd-tet) that bind Bacillus anthracis spores
cctgcgcgtgatccggttaatatg

SEQ ID NO: 147 - DNA, 24 bp, ARTIFICIAL SEQUENCE
  MAT_PEPTIDE: Source of material Sequenced peptides isolated from phage display library (fd-tet) that bind Bacillus anthracis spores
gttgctcggagtacgggtgattcg

SEQ ID NO: 148 - DNA, 24 bp, ARTIFICIAL SEQUENCE
  MAT_PEPTIDE: Source of material Sequenced peptides isolated from phage display library (fd-tet) that bind Bacillus anthracis spores
gttgataggggttcggctacttcg

SEQ ID NO: 149 - DNA, 24 bp, ARTIFICIAL SEQUENCE
  MAT_PEPTIDE: Source of material Sequenced peptides isolated from phage display library (fd-tet) that bind Bacillus anthracis spores
gttgatcggggtactactttgtcg

SEQ ID NO: 150 - DNA, 24 bp, ARTIFICIAL SEQUENCE
  MAT_PEPTIDE: Source of material Sequenced peptides isolated from phage display library (fd-tet) that bind Bacillus anthracis spores
gttgatcggtcttctactactacg

SEQ ID NO: 151 - DNA, 24 bp, ARTIFICIAL SEQUENCE
  MAT_PEPTIDE: Source of material Sequenced peptides isolated from phage display library (fd-tet) that bind Bacillus anthracis spores
gtggatcgtactcctccttcgcag

SEQ ID NO: 152 - DNA, 24 bp, ARTIFICIAL SEQUENCE
  MAT_PEPTIDE: Source of material Sequenced peptides isolated from phage display library (fd-tet) that bind Bacillus anthracis spores
gtggatcgtacgtcgtctccggcg

SEQ ID NO: 153 - DNA, 24 bp, ARTIFICIAL SEQUENCE
  MAT_PEPTIDE: Source of material Sequenced peptides isolated from phage display library (fd-tet) that bind Bacillus anthracis spores
gtggggcgtgctaatccttcgtcg

SEQ ID NO: 154 - DNA, 24 bp, ARTIFICIAL SEQUENCE
  MAT_PEPTIDE: Source of material Sequenced peptides isolated from phage display library (fd-tet) that bind Bacillus anthracis spores
gtggggcgtgaggggggggcggtg

SEQ ID NO: 155 - DNA, 24 bp, ARTIFICIAL SEQUENCE
  MAT_PEPTIDE: Source of material Sequenced peptides isolated from phage display library (fd-tet) that bind Bacillus anthracis spores
gtgaatcagacggcgcagcctgcg

SEQ ID NO: 156 - DNA, 24 bp, ARTIFICIAL SEQUENCE
  MAT_PEPTIDE: Source of material Sequenced peptides isolated from phage display library (fd-tet) that bind Bacillus anthracis spores
gtgccgccgccttcttcttcgacg

SEQ ID NO: 157 - DNA, 24 bp, ARTIFICIAL SEQUENCE
  MAT_PEPTIDE: Source of material Sequenced peptides isolated from phage display library (fd-tet) that bind Bacillus anthracis spores
gtgcctcgtccggatgctacttcg

SEQ ID NO: 158 - DNA, 24 bp, ARTIFICIAL SEQUENCE
  MAT_PEPTIDE: Source of material Sequenced peptides isolated from phage display library (fd-tet) that bind Bacillus anthracis spores
gttcctacgcgtactcctcagggg

SEQ ID NO: 159 - DNA, 24 bp, ARTIFICIAL SEQUENCE
  MAT_PEPTIDE: Source of material Sequenced peptides isolated from phage display library (fd-tet) that bind Bacillus anthracis spores
gttcctacgtctcgtgcggatcag

SEQ ID NO: 160 - DNA, 24 bp, ARTIFICIAL SEQUENCE
  MAT_PEPTIDE: Source of material Sequenced peptides isolated from phage display library (fd-tet) that bind Bacillus anthracis spores
gtgcctactacgcgtgagacttcg

SEQ ID NO: 161 - DNA, 24 bp, ARTIFICIAL SEQUENCE
  MAT_PEPTIDE: Source of material Sequenced peptides isolated from phage display library (fd-tet) that bind Bacillus anthracis spores
gttcagcctactgcggctccgccg

SEQ ID NO: 162 - DNA, 24 bp, ARTIFICIAL SEQUENCE
  MAT_PEPTIDE: Source of material Sequenced peptides isolated from phage display library (fd-tet) that bind Bacillus anthracis spores
gttcgtccgactccgactgatacg

SEQ ID NO: 163 - DNA, 24 bp, ARTIFICIAL SEQUENCE
  MAT_PEPTIDE: Source of material Sequenced peptides isolated from phage display library (fd-tet) that bind Bacillus anthracis spores
gtgagtgatcggggtactgcgacg

SEQ ID NO: 164 - DNA, 24 bp, ARTIFICIAL SEQUENCE
  MAT_PEPTIDE: Source of material Sequenced peptides isolated from phage display library (fd-tet) that bind Bacillus anthracis spores
gtttcggatcgggtttctcctgcg

SEQ ID NO: 165 - DNA, 24 bp, ARTIFICIAL SEQUENCE
  MAT_PEPTIDE: Source of material Sequenced peptides isolated from phage display library (fd-tet) that bind Bacillus anthracis spores
gtgtctcctacgcagcagcagacg

SEQ ID NO: 166 - DNA, 24 bp, ARTIFICIAL SEQUENCE
  MAT_PEPTIDE: Source of material Sequenced peptides isolated from phage display library (fd-tet) that bind Bacillus anthracis spores
gttagtcagcctgcttctccttcg

SEQ ID NO: 167 - DNA, 24 bp, ARTIFICIAL SEQUENCE
  MAT_PEPTIDE: Source of material Sequenced peptides isolated from phage display library (fd-tet) that bind Bacillus anthracis spores
gtgtctagggaggcggctgcgtcg

SEQ ID NO: 168 - DNA, 24 bp, ARTIFICIAL SEQUENCE
  MAT_PEPTIDE: Source of material Sequenced peptides isolated from phage display library (fd-tet) that bind Bacillus anthracis spores
gtgtcgcggattccgagtgagacg

SEQ ID NO: 169 - DNA, 24 bp, ARTIFICIAL SEQUENCE
  MAT_PEPTIDE: Source of material Sequenced peptides isolated from phage display library (fd-tet) that bind Bacillus anthracis spores
gtgagtaggatggagtctactccg

SEQ ID NO: 170 - DNA, 24 bp, ARTIFICIAL SEQUENCE
  MAT_PEPTIDE: Source of material Sequenced peptides isolated from phage display library (fd-tet) that bind Bacillus anthracis spores
gtttctcgtactcaggagcatgtg

SEQ ID NO: 171 - DNA, 24 bp, ARTIFICIAL SEQUENCE
  MAT_PEPTIDE: Source of material Sequenced peptides isolated from phage display library (fd-tet) that bind Bacillus anthracis spores
gtgtctacgcggccgacggagacg

SEQ ID NO: 172 - DNA, 24 bp, ARTIFICIAL SEQUENCE
  MAT_PEPTIDE: Source of material Sequenced peptides isolated from phage display library (fd-tet) that bind Bacillus anthracis spores
gtgactgatgcgcgtacgccggcg

SEQ ID NO: 173 - DNA, 24 bp, ARTIFICIAL SEQUENCE
  MAT_PEPTIDE: Source of material Sequenced peptides isolated from phage display library (fd-tet) that bind Bacillus anthracis spores
gtcactaatgcgaatagtccgtcg

SEQ ID NO: 174 - DNA, 24 bp, ARTIFICIAL SEQUENCE
  MAT_PEPTIDE: Source of material Sequenced peptides isolated from phage display library (fd-tet) that bind Bacillus anthracis spores
gttactcctcgtgctgatagtacg

SEQ ID NO: 175 - DNA, 24 bp, ARTIFICIAL SEQUENCE
  MAT_PEPTIDE: Source of material Sequenced peptides isolated from phage display library (fd-tet) that bind Bacillus anthracis spores
gttacgcgtgatctttcgagttcg

SEQ ID NO: 176 - DNA, 24 bp, ARTIFICIAL SEQUENCE
  MAT_PEPTIDE: Source of material Sequenced peptides isolated from phage display library (fd-tet) that bind Bacillus anthracis spores
gtgacgagggggtcgatgaatacg

SEQ ID NO: 177 - DNA, 24 bp, ARTIFICIAL SEQUENCE
  MAT_PEPTIDE: Source of material Sequenced peptides isolated from phage display library (fd-tet) that bind Bacillus anthracis spores
gtgacgcggaatcctgctgcgtcg

SEQ ID NO: 178 - DNA, 24 bp, ARTIFICIAL SEQUENCE
  MAT_PEPTIDE: Source of material Sequenced peptides isolated from phage display library (fd-tet) that bind Bacillus anthracis spores
gttacgaggaatccttcggatgcg

SEQ ID NO: 179 - DNA, 24 bp, ARTIFICIAL SEQUENCE
  MAT_PEPTIDE: Source of material Sequenced peptides isolated from phage display library (fd-tet) that bind Bacillus anthracis spores
gttacgcgtaatacgagtgcgtcg

SEQ ID NO: 180 - DNA, 24 bp, ARTIFICIAL SEQUENCE
  MAT_PEPTIDE: Source of material Sequenced peptides isolated from phage display library (fd-tet) that bind Bacillus anthracis spores
gtgactagtgcttcttcttcgcag

SEQ ID NO: 181 - DNA, 24 bp, ARTIFICIAL SEQUENCE
  MAT_PEPTIDE: Source of material Sequenced peptides isolated from phage display library (fd-tet) that bind Bacillus anthracis spores
gttgttagggagcctacgcattcg

SEQ ID NO: 182 - DNA, 24 bp, ARTIFICIAL SEQUENCE
  MAT_PEPTIDE: Source of material Sequenced peptides isolated from phage display library (fd-tet) that bind Bacillus anthracis spores
gtggtgaggggttcggatggtgcg

(19) United States
(12) Patent Application Publication
(10) Pub. No.: US 2007/0037230 A1
(43) Pub. Date: Feb. 15, 2007

(54) Peptides for selectively and specifically binding Bacillus anthracis spores and use thereof in landscape phages

(76) Inventors: Valery A. Petrenko, Auburn, AL (US); Jennifer R. Brigati, Auburn, AL (US); Iryna B. Sorokulova, Auburn, AL (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/414,683

(22) Filed: Apr. 28, 2006

Related U.S. Application Data
(60) Provisional application No. 60/676,661, filed on Apr. 29, 2005.

(51) Int. Cl. G01N 33/554; C07K 16/12; C07K 14/32

(52) U.S. Cl. 435/7.32; 530/388.4; 530/350

(57) ABSTRACT

Several peptides for selectively and specifically binding Bacillus anthracis spores are disclosed herein. It is contemplated that the isolated peptides will be incorporated into diagnostic probes. The diagnostic probes may include landscape page probes and antibody probes, as well as any other probes known in the art. Inventors contemplate that the probes could be further used in real-time continuous monitoring of the environment so that detection systems for monitoring the low concentrations of Bacillus anthracis spores may be developed.

### CROSS-REFERENCE TO RELATED APPLICATIONS

This application relates to and claims priority from U.S. Provisional Application Ser. No. 60/676,661 filed Apr. 29, 2005.

### STATEMENT REGARDING FEDERALLY SPONSORED RESEARCH OR DEVELOPMENT

This work was supported by Army Research Office/Defense Advance Research Projects Agency Grant DAAD19-01-10454, NIH Grant NIH-1 R21AI055645, DARPAMDA9272-01-1-0030 and ARO DAAD19-00-1-0032.

### INCORPORATION-BY-REFERENCE OF MATERIAL SUBMITTED ON A COMPACT DISC

Not Applicable

### BACKGROUND AND SUMMARY

The present application is directed to peptides that have selectivity and specificity in binding Bacillus anthracis spores. When the peptides described herein are incorporated into a landscape phage, diagnostic probes with high precision inaccuracy are achieved.

Spores of Bacillus anthracis, the causative agent of anthrax, have been used in successful bioterrorism attacks in the United States. In instances of bioterrorism using anthrax, rapid recognition of exposure to Bacillus anthracis is essential to allow early initiation of antibiotic treatment, which can greatly reduce mortality. Detection of Bacillus anthracis spores before the onset of symptoms in the victims requires a diagnostic probe having selectivity and specificity in binding the spores and requires high precision and accuracy to avoid false alarms.

It is challenging to detect Bacillus anthracis spores because several closely related Bacillus species are ubiquitous in the environment. Bacillus cereus, is an opportunistic human pathogen while Bacillus thuringiensis often used as an insectide. Both of these Bacillus species have close genetic similarieies to Bacillus anthracis. Other related Bacillus species are also present in the environment, including Bacillus suttilis and Bacillus licheniformis, both antibiotic producers and Bacillus megaterium, an animal pathogen. In order to avoid costly false alarms, a detection system incorporating a diagnostic probe must be sensitive enough to detect low concentrations of Bacillus anthracis spores, but also must be selective enough to differentiate between Bacillus anthracis and the closely related species such as Bacillus cereus and Bacillus thuringiensis and other Bacillus species.

The most effective system for monitoring the air for spores would be a continuous monitoring system, and none of the assays currently available for detection of Bacillus anthracis to date have been adapted for real-time, continuous monitoring of the environment. While immunoassay and biosensor based detection systems are the best prospect for continuous monitoring systems, such systems require specific, selectable and stable diagnostic probes to detect the Bacillus anthracis spores. It is known that antibodies are diagnostic probes that may be used for this purpose. However, these traditional diagnostic probes are not as robust as necessary for continuous monitoring of the environment.

The present application describes the use of specific peptides incorporated into a landscape phage probe, wherein the peptides form a dense organic landscape on the surface of the phage, and allow for selective and specific binding of Bacillus anthracis spores with a high degree of accuracy and precision.

Phage-display libraries refer to a selection technique wherein a library of variants of a peptide or protein is expressed on the outside of a phage virion, while the genetic material encoding the peptide or protein remains inside the phage. Phage-display libraries are constructed by the genetic modification of filamentous bacterial viruses (phages) such as M13, f1, and fd. The outer coats of these filamentous phages are composed of thousands of α-helical subunits of major coat protein pVIII, (see, e.g. FIG. 4) which form a tube encasing the viral DNA. At the tips of the phage are several copies of each of the minor proteins, pIII, pVI, pvII, and pIX. To create a phage-display library, degenerate synthetic oligonucleotides are spliced in-frame into one of the phage coat protein genes, so that the peptide encoated by the degenerate oligonucleotide is fused to the coat protein and thereby displayed on the exposed surface of the phage virion. Accordingly, each phage virion displays multiple copies of one particular peptide.

In landscape phages, as in traditional phage-display constructs, foreign peptides or proteins are fused to coat proteins on the surface of the virus particle. Unlike conventional phage constructs, however, landscape phages display thousands of copies of the peptide in a repeating pattern, comprising a major fraction of the viral surface, FIG. 4. The phage body serves as an interacting scaffold to constrain the peptide into a particular confirmation, creating a defined organic surface structure, i.e., the landscape. The particular conformation, and thus organic surface structure, varies from one phage clone to the next Accordingly, a landscape phage library is a huge population of such phages, encompassing billions of clones with different surface structures and biophysical properties.

A landscape phage library was used in conjunction with the present application to discover particular peptides having selectivity and specificity in binding Bacillus anthracis spores. The landscape phage library used in conjunction with the present application contained random eight amino acid peptides fused to all 4,000 copies of the major protein pVIII of the virus fd-tet. The wild type fd-tet vector was modified to have a 2,775 base pair BglII fragment of transposon Tn10 inserted into the BamHI site of the wild-type virus genome. The resulting recombinant molecule is 9,183 base pairs in size and is designated f8-1. FIG. 1 demonstrates the genome of the f8-1 bacteriophage with genes marked I through XI. This single stranded viral DNA has 9,183 nucleotides that are numbered clockwise with unique Hinc II site located in gene II (represented by zero). FIG. 2 sets forth the nucleotide sequence of the f8-1 bacteriophage DNA. The organism is designated as the filamentous phage display vector f8-1, molecular type is genomic DNA, and the lab host is E. coli. In FIG. 2, gene VIII, at base pairs 1301-1522, are depicted in bold and designate the nucleotide sequence that codes for a peptide that is displayed as a major coat protein on the viral surface of the phage.

The insertion of the Tn10 fragment into the wild-type fd virus genome disrupts the minus-strand origin of replication. This disruption greatly reduces the intracellular copy number of circular, double stranded RF DNA without greatly reducing phage yield. As a result, the fd-tet mutants that are completely effective for assembly are propagated, whereas mutants in other strains of filamentous phage in which the Tn10 fragment has not been inserted at the specific loci in the fd genome kill the host without yielding progeny particles, a phenomenon known as cell killing. The absence of cell killing in fd-tet has an important advantage, namely, that partial defects in coat protein function due to insertion of foreign peptides or protein domains are better tolerated than in the wild type phage, reducing selective pressure for loss or alteration of the insert.

The resulting f8-1 bacteriophage vector is a filamentous phage display vector that displays foreign peptides on all 4,000 coat protein pVIII molecules in a phage. As previously mentioned, the f8-1 vector was constructed by engineering three single base pair substitutions into filamentous phage cloning vector fd-tet (GenBank Accession No. AF217317). FIG. 3 demonstrates the segment of the f8-1 vector DNA, base pairs 1367-1397 that contain the Pst I and Bam HI cloning sites. The signal peptidase cleaves the pre-coat proteins between A-1 and A1.

The f8-1 vector is very similar in most important characteristics to its fd-tet parent. The f8-1 vector operates as a phage display vector and allows a short degenerate coding sequence to be inserted between the Pst I and the Bam HI cloning site to create a library of phage displaying different random peptides at the N-terminus of all of the approximately 4,000 copies of the major coat protein pVIII. The structure of a landscape phage incorporating the f8-1 vector is demonstrated in FIG. 4. FIG. 4A demonstrates the nucleotide and amino acid sequences corresponding to the beginning of the mature form protein pVIII and the vector f8-1 and the recombinant protein pVIII in the library. Only the viral strand of DNA (anti-complementary to mRNA) is shown. In FIG. 4B, the short length of the phage surface is modeled. The peptide inserts are depicted with dark atoms, while the wild type peptides are displayed as light colored atoms. The overall arrangement of the peptide inserts and wild-type peptides is fixed by virion symmetry.

It is known that landscape phages, such as the phage vector f8-1, may serve as substitutes for antibodies against various agents and receptors, including live bacterial cells. Landscape phage probes have been used in enzyme linked imunosorbent assays (ELISA) and thickness shear mode quartz sensors to detect antigens. Use of landscape phages as substitutes for antibodies has several advantages: Landscape phages produce up to 4,000 copies of the binding peptide on their surfaces, allowing multivalent interactions with a target antigen; phages can be produced rapidly and inexpensively in large quantities; landscape phages are resistant to heat, organic solvents, and many other stresses; landscape phages may be stored indefinitely at moderate temperatures without loss of activity, or at 37° Celsius with only minimal loss of activity after seven months.

Accordingly, the present application sets forth specific peptides that selectively and specifically bind Bacillus anthracis, and that may be incorporated into diagnostic probes, such as landscape phage probes or antibody probes to provide selective binding to Bacillus anthracis spores.

### SUMMARY OF THE INVENTION

The present application discloses isolated peptides having selective activity and specificity in binding Bacillus anthracis spores. It is contemplated that the isolated peptides will be incorporated into diagnostic probes. The diagnostic probes may include landscape phage probes and antibody probes, as well as any other probes known in the art.

The isolated peptides are eight amino acids in length and are preferably selected from the group: AGRAGGGV, AGRGPGLP, ANRVPPTS, ATRPASSM, DARGTTHM, EPRLSTHS, ETRETHGA, VDRGSATS, VDRGTTLS, VPRPDATS, VSDRGTAT, VSRIPSET, VTRGSMNT, EPRAPSL, EPKPHTFS, EPHPKTST, DRTGATLT, EKTPVTAT, ERTVATTQ, VSQPASPS, VTRNTSAS.

### BRIEF DESCRIPTION OF THE SEVERAL VIEWS OF THE DRAWINGS

FIG. 1 is a graphical representation of a genome of a f8-1 bacterial phage;

FIG. 2 is a nucleotide sequence of vector f8-1 DNA base pairs 1 through 9181 with gene VIII at base pairs 1301 through 1522 depicted in bold;

FIG. 3 is a graphical depiction of sequences of the cloning sites and the vector f8-1, particularly the PstI and BamHI cloning sites;

FIG. 4 demonstrates the structure of the f8-1 landscape phage, with FIG. 4A depicting the nucleotide and amino acid sequences corresponding to the beginning of the mature form of major coat protein pVIII in vector f8-1 the recombinant pVIII in the library where n represents an equal mixture of G, A, T and C, and k an equal mixture of G and T with only the viral strand of DNA (anticomplimentary to mRNA) shown, while FIG. 4B demonstrates a computer model of a short length of the phage surface with inserted peptides shown as dark atoms and wild-type peptides shown as light atoms;

FIG. 5 is a bar graph demonstrating phage recovery during four rounds of selection as further detailed, herein;

FIG. 6 is a bar graph demonstrating the binding affinity of selected phages containing particular peptides having amino acid sequences demonstrated in FIG. 6 to Bacillus anthracis immobilized on a microtiter plate;

FIG. 7 is a bar graph demonstrating the binding of biotinylated Bacillus anthracis spores to selected and control phages immobilized on a microtiter plate containing the particular peptides having the amino acid sequences demonstrated in FIG. 7;

FIG. 8 is a bar graph demonstrating the binding of non-biotinylated Bacillus anthracis spores to selected and control phage immobilized on a microtiter plate;

FIG. 9 is a series of bar graphs (A-F) demonstrating the selective bindings of landscape phage clones having particular peptides incorporated therein to Bacillus anthracis spores in a coprecipitation assay;

FIG. 10 is a bar graph demonstrating binding of selected phage clones including specific peptides having the amino acid sequences demonstrated in FIG. 10 to Bacillus anthracis spores as determined by phage capture ELISA;

FIG. 11 is a chart demonstrating grouping of amino acid sequences identified herein into specific families based on common motifs.

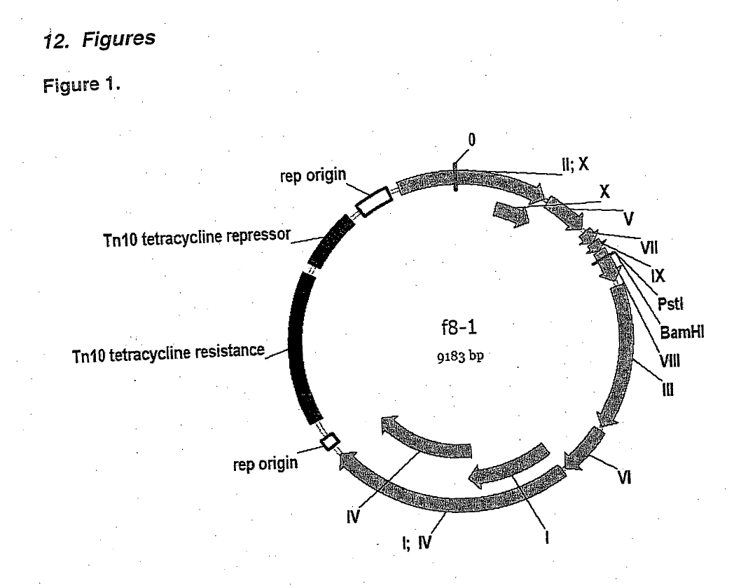

### DETAILED DESCRIPTION OF THE INVENTION

The present application describes a method to provide peptides having selectivity and specificity in binding Bacillus anthracis spores. It is contemplated that the peptides identified herein may be incorporated into diagnostic probes that are well known in the art including, but not limited to, landscape phage probes and antibody probes.

The present inventors researched evaluating the prospect of using not only individual peptides, but phages themselves as the basis for the development of a robust and inexpensive diagnostic probe. Landscape phage clones were identified from a phage clone library that bind to Bacillus anthracis spores. The specificity and selectivity of the peptides associated with the identified phage clones were studied for specificity and selectivity of their interaction with the target selector spores in comparison with other Bacillus species. As detailed herein, the inventors contemplate incorporating the peptides having specificity and selectivity for Bacillus anthracis spores into a diagnostic probe that may be used in monitoring devices.

Such monitoring devices are preferably real time detectors that allow for automatic, continuous biological monitoring of an environment. For use in human workplace environments, the real-time detector may be coupled to a air-tube-liquid concentrator. The central element of a real time detector is a probe that binds bacterium, spore, virus or toxin and, as part of an analytical platform, generates a measurable signal. The platform measures changes in mass, frequency of oscillation, capacitance, resistance, surface plasmon resonance, reflectometric interference, fluorescence, or other effects caused by interaction of the probe with the biological threat agent. More sophisticated platforms, such as electrochemiluminescence (ECL) enzymed-linked imunosorbent assay (ELISA) and other amino-sandwich platforms, use two different probes: capture probes and detector or reporter, tracer probes. The choice of the detection probes is dictated by their specificity, selectively, performance, storage, operational and environmental stability. In most known platforms, the probes are polyclonal or monoclonal antibodies. However, the inventors contemplate using landscape phage probes are substitutes for such antibodies in the above described systems to provide a unique monitoring system for Bacillus anthracis spores.

Selection of phages from the landscape phage display library that bind to Bacillus anthracis spores is accomplished using the landscape phage display library f8/8, further described in the Materials and Methods portion. The landscape library was constructed by splicing degenerate oligonucleotides into gene pVIII of the f8-1 vector so that foreign random peptides were displayed as the end terminal protein of the major coat protein pVIII. From this library, phage clones that bond to Bacillus anthracis spores were selected through a panning procedure in which the phage library was incubated with immobilized Bacillus anthracis spores, non-bound phages were washed away, and bound phages were eluted with a mild acid.

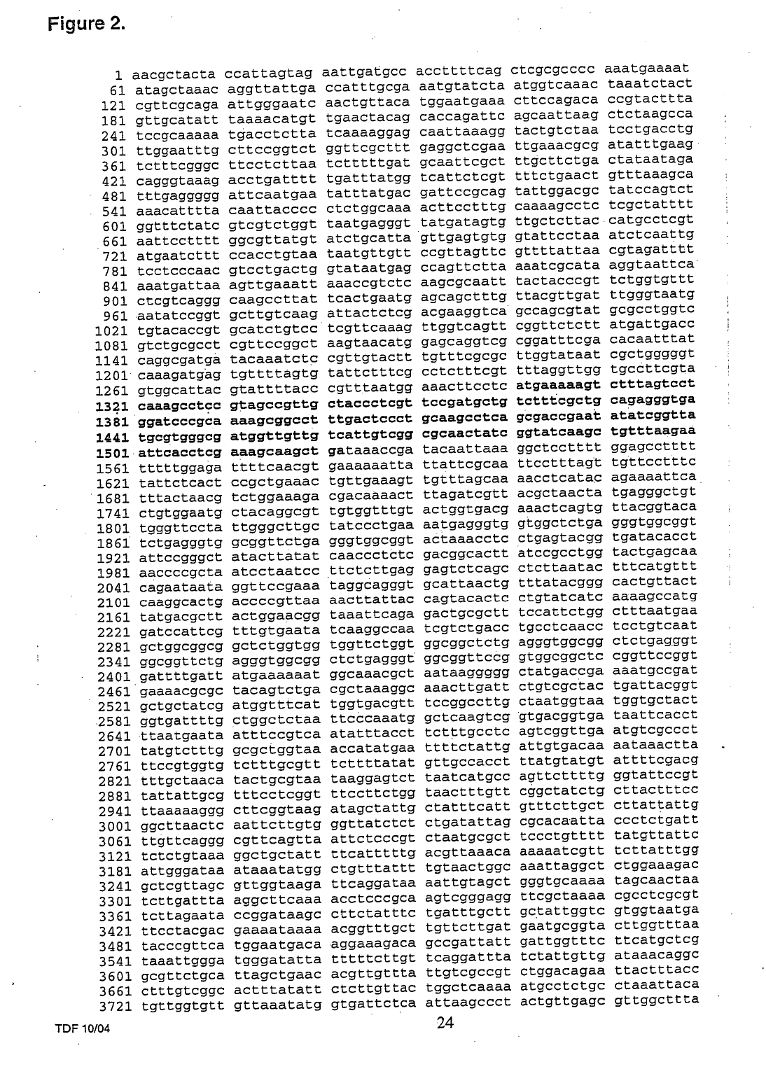

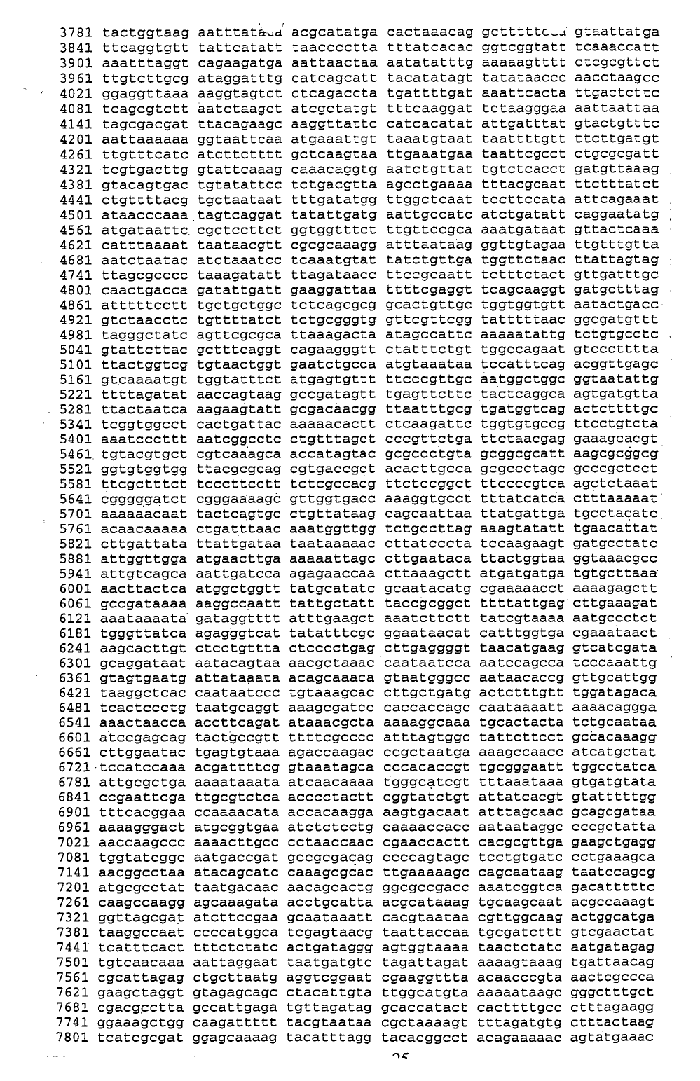

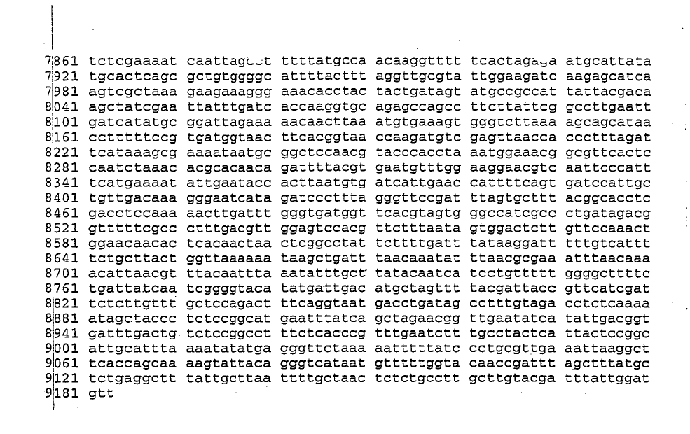

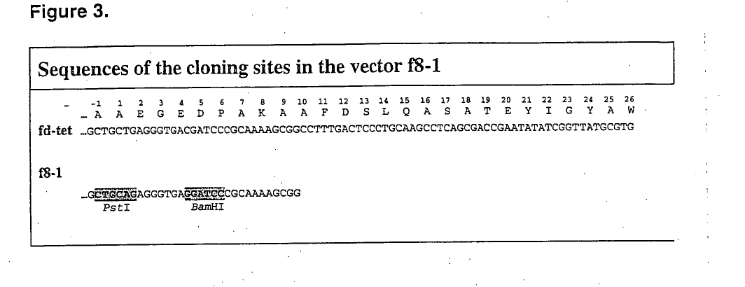

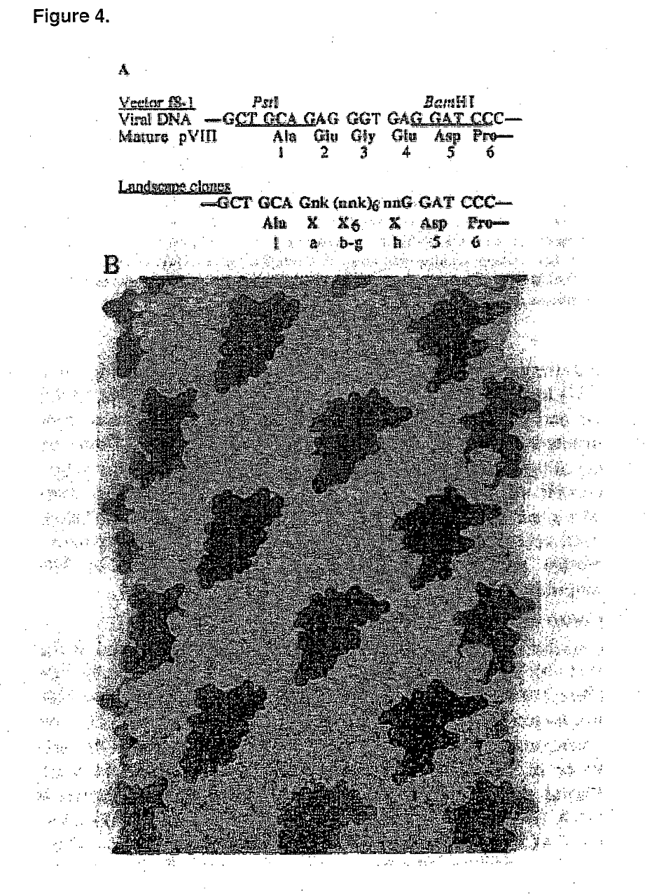

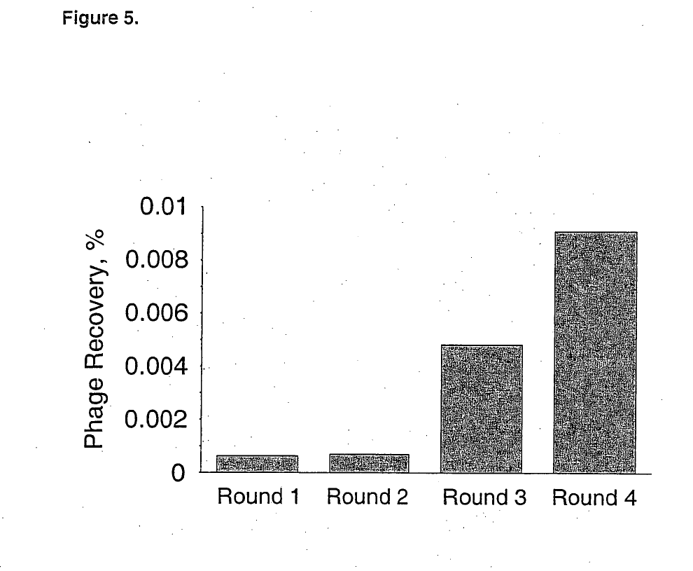

Phages that bound to spores in the initial selection procedures were amplified and used as input in subsequent rounds of selection. Four rounds of selection occurred in total. Numbers of ineffective phage particles present in the input, washes, and eluate each round of selection were determined by titering. As demonstrated in FIG. 5, the increase in phage recovery after each round of selection indicated an increase in the representation of phage clones in the initial selection procedure that were capable of binding to Bacillus anthracis spores. After four rounds of selection, sixteen randomly picked clones were isolated, and the segment of genomic DNA encoding the displayed peptide (readily determined through FIG. 2) was sequenced.

Additional Bacillus anthracis binding phage probes were identified using a biased selection procedure in which the library was first depleted of phage clones which bind to common Bacillus antigens. To accomplish this, the phage library was mixed with Bacillus cereus spores and incubated for 30 minutes on a rotator. The spore/phage solution was then centrifuged to pellet the spores in any spore bound phage. The supernatant (depleted library) was transferred to a new tube and further depleted against Bacillus thuringiensis and Bacillus subtilis before use in a biopanning procedure.

The panning selection procedure and the biased selection procedure revealed 91 peptides specifically binding spores of Bacillus anthracis. The amino acid sequences of the 91 phage formed peptides isolated in both selection procedures are shown, below, in Table 1.

```
  1. AARQPAGM
  2. AARQPMAS
  3. AARSVTDS
  4. ADRVPYPS
  5. AGRAGGGV
  6. AGRGPGLP
  7. AHREMPQG
  8. ANRVPPTS
  9. ARPSDGLS
10. ARSAGPLP
11. ARSALPSS
12. ARSNPALT
13. ARSQPALS
14. ARSSPSLA
15. ASRDGAVM
16. ASRSSGAL
17. ASRTSGLP
18. ATRPASSM
19. ATRTAPTS
20. ATRTTSPM
21. AVRDQPNL
22. DARGTTHM
23. DDAGRGTP
24. DKLSSSGT
25. DLGDRSQG
26. DPRAAVTA
27. DPSAYSRA
28. DRTGATLT
29. DSRALIAP
30. DTRETGSS
31. DYDAVMRT
32. DYPTKTNS
33. EDGRGGTA
34. EDRVFPST
35. EERQNPSG
36. EFTARPSS
37. EKTPVTAT
38. ENSRSTQM
39. EPHPKTST
40. EPKMQAAQ
41. EPKPHTFS
42. EPMRDMAS
43. EPRAPASL
44. EPRLAGDQ
45. EPRLSPHS
46. EPSRGPGS
47. ERAATDQT
48. ERTVATTQ
49. ESRGVGTE
50. ETRVPHGA
51. EYERASQG
52. GLRTTPNT
53. GPESQTAS
54. GQRTPPTT
55. PARDPVNM
56. VARSTGDS
57. VDRGSATS
58. VDRGYFLS
59. VDRSSTTT
60. VDRTPPSQ
61. VDRTSSPA
62. VGRANPSS
63. VGREGGAV
64. VNQTAQPA
65. VPPPSSST
66. VPRPDATS
67. VPTRTPQG
68. VPTSRADQ
69. VPTTRETS
70. VQPTAAPP
71. VRPTPTDT
72. VSDRGTAT
73. VSDRVSPA
74. VSPTQQQT
75. VSQPASPS
76. VSREAAAS
77. VSRIPSET
78. VSRMESTP
79. VSRTQEHV
80. VSTRPTET
81. VTDARTPA
82. VTNANSPS
83. VTPRADST
84. VTRDLSSS
85. VTRGSMNT
86. VTRNPAAS
87. VTRNPSDA
88. VTRNTSAS
89. VTSASSSQ
90. VVREPTHS
91. VVRGSDGA
```

It is noted that the amino acid sequence of the peptides listed in Table 1 are presented in alphabetical order. A three letter abbreviation designator for these peptides is provided in the Sequence Listing below, as well as the nucleotide sequences of the peptides. In all instances, numbering of the sequences are consistent.

As demonstrated in FIG. 11, the peptides identified in the selection procedures may be assembled into families with particular motif or consensus sequences. The characteristic motifs of the phage formed peptides binding Bacillus anthracis spores were determined using the RELIC program developed by Mandaya et al 2004 available at http://relic.bio.anL.gov/index.aspx.

Table 2 demonstrates how families of peptides may be arranged based on common motifs:

TABLE 2
Amino acid sequences of peptides carried by phage selected in the
non-biased procedure, with bold italics indicating common motifs.

| Family 1 | Family 2 | Family 3 |
|---|---|---|
| [symbol] | [symbol] | [symbol] |
| [symbol] | [symbol] | [symbol] |
| [symbol] | [symbol] | |
| [symbol] | | |
| [symbol] | | |
| [symbol] | | |

As demonstrated in Table 2, family 1, with six members, was characterized by the presence of a negatively charged amino acid (E or D) at the first position, usually a proline residue at the second position and a positively charge amino acid (R, K or H) at the third position. Another interesting feature of this family was the frequent presence of a migrating dipeptide PH, which was replaced by PK in peptide 4. Family 2, with three members, contained the consensus sequence (D/E) (R/K) TXATXT. Family 3, with two members, contained the consensus sequence V(S/T)XXXSXS.

After the selection procedures, the identified peptides were tested for specifically in biding Bacillus anthracis. Specificity is herein defined as the ability of the recombinant phage to interact with spores as a result of the presence of a specific peptide sequence expressed by the phage. To determine specificity, binding of the selected phage clones displaying the identified peptides were compared with the binding of a wild type phage to Bacillus anthracis. The selected phage clones were further compared with a non-related recombinant phage from the landscape library for binding activity.

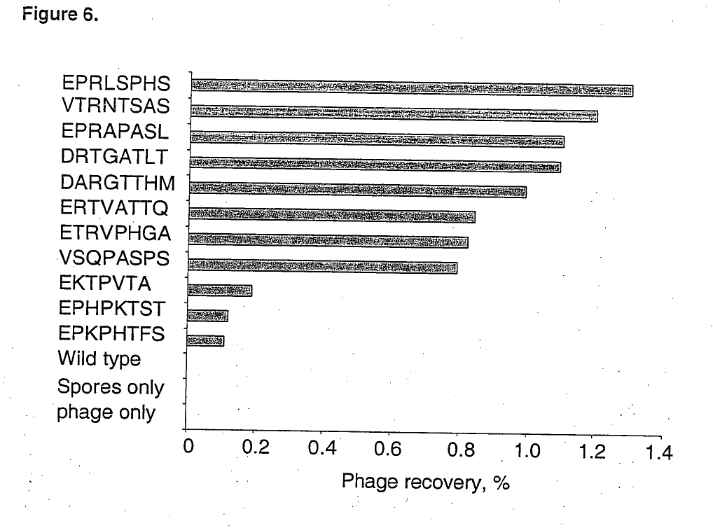

The relative binding of the isolated phage clones to Bacillus anthracis spores was measured by a phage-capture assay and an ELISA. In the phage-capture assay, a procedure very similar to the selection procedure was used to determine relative binding of the phage clones to immobilized Bacillus anthracis spores. Briefly, selected phage clones were added to the wells of a microtiter plate that were coated with Bacillus anthracis spores. After an incubation to allow binding, non-bound phage were washed away and bound phages were eluted and titered. The percentage recovery was determined as the ratio of the eluted phages to input phages. As shown in FIG. 6, selected clones bind at a much higher percentage than wild type phage to Bacillus anthracis spores in this assay.

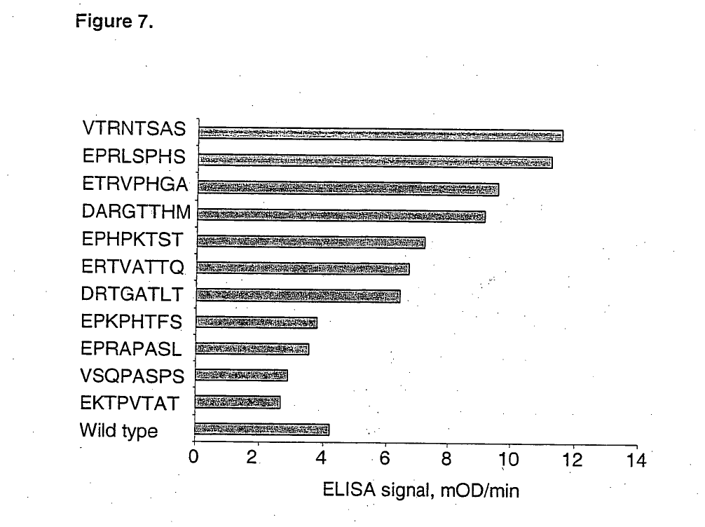

In the ELISA, wells of a microtiter plate were coated with phage and then incubated with biotinylated Bacillus anthracis spores. Alkaline phosphatase conjugated to streptavivin was added to the wells microtiter plate to bind to the biotinylated spores, and p-nitrophenylphosphate was used to detect the binding. As shown in FIG. 7, many of the isolated phage clones bound to Bacillus anthracis spores had a higher level than wild type phage. Some clones bound strongly to Bacillus anthracis in both assays, whereas other clones gave inconsistent results between the two assays. This is not completely unexpected because in the ELISA phage were fixed to the plate and spores were captured from solution, whereas in the phage-capture assay, spores were fixed to the plate and phage were bound from solution. Thus, in these tests, phage could adopt different confirmations, allowing monovalent or multivalent interactions with spore receptors.

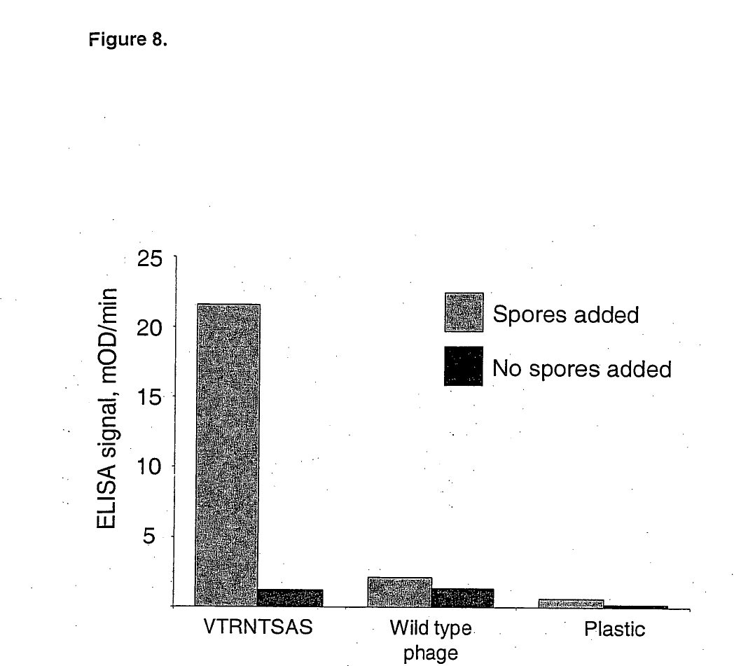

To confirm that the ELISA results were not attributable to biotinylation of contaminants of the spore preparation, an ELISA was done in another format, with antibodies specific for Bacillus anthracis spores. FIG. 8 exemplifies specific binding of phages carrying the peptide having amino acid sequence of VTRNTSAS to spores, revealed with a monoclonal antibody BD8. It is clear from this experiment that phages were capturing spores and not some other contaminants of the spore preparation.

After the selectivity tests where performed, tests to determine the selectivity of particular peptides Bacillus anthracis versus other Bacillus species were performed. As used herein, selectivity is defined as the ability of a recombinant phage clone to preferentially interact with the selector in comparison with other potential targets. To determine the selectivity of phage probes for the selector Bacillus anthracis spores versus spore of other Bacillus species, a coprecipitation assay was used. Phage displaying the peptides having amino acid sequences DARGTTHM, EPRLSPHS, and VTRNTSAS were initially examined because of their high binding in both the ELISA and the phage-capture assays for specificity, see FIGS. 6 and 7. Phage displaying the peptides having amino acid sequences DRTGATLT and EPRAPASL were tested because of the high binding they demonstrated in the phase capture assay, and the phage carrying the peptide ETRVTHGA were tested because of the high binding they demonstrated in the ELISA.

In the coprecipitation assay, these phages were mixed individually with spores of various Bacillus species. After incubating, spores were collected by low speed centrifugation, so that only phages bound to spores would be found in the pellet. Phages without spores were not used as control to ensure that the phage were not aggregating and precipitating on their own.

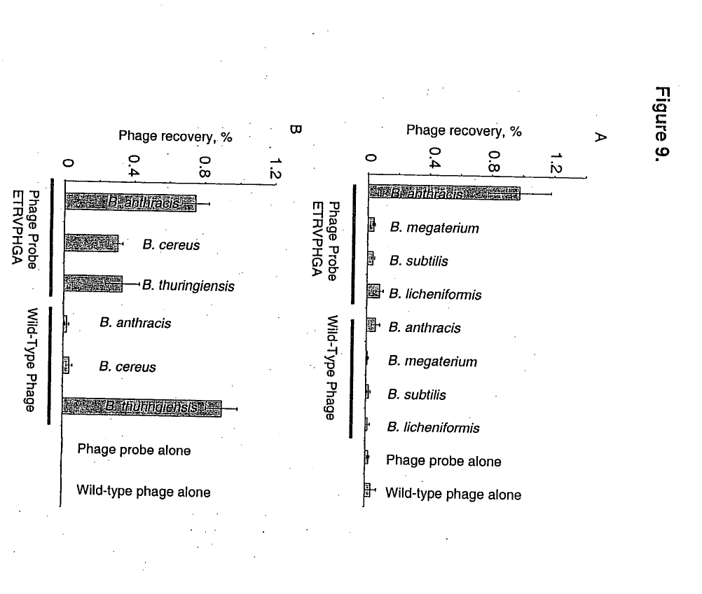

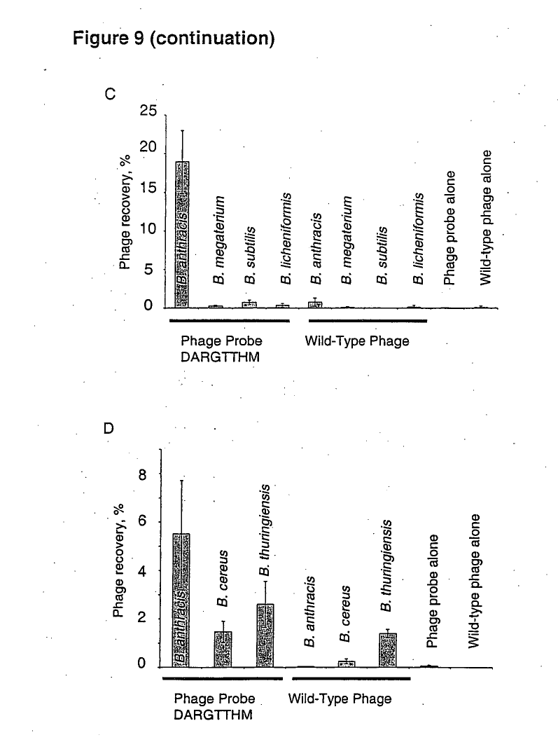

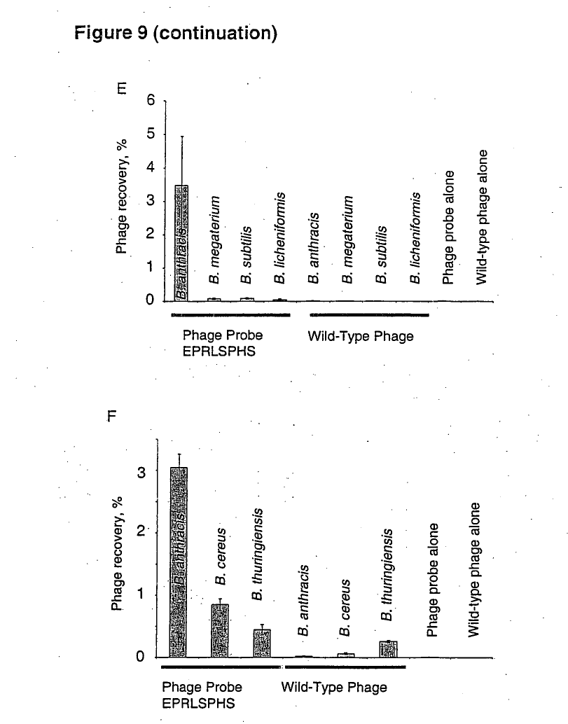

Initial tests were done with distant relatives of Bacillus anthracis: Bacillus megaterium, Bacillus subtilis and Bacillus licheniformis. Some clones exhibited very low binding to these distant relatives, while others bound them nearly as well as Bacillus anthracis. It was revealed that the phage carrying the peptide having the sequence DARGTTHM bound to Bacillus anthracis, 75 fold better than to Bacillus megaterium, 25 fold better than to Bacillus subtilis, and 50 fold better than to Bacillus licheniformis. Likewise, phage carrying the peptide having the amino acid sequence EPRLSPHS bound to Bacillus anthracis 43 fold better than to Bacillus megaterium, 39 fold better than to Bacillus subtilis, and 70 fold better than to Bacillus licheniformis. Phage carrying the peptide having amino acid sequence ETRVPHGA bound to Bacillus anthracis 24 fold better than to Bacillus megaterium, 24 fold better than to Bacillus subtilis, and 12 fold better than to Bacillus licheniformis. A graphical comparison of the relative binding interactions of the three peptides described above are demonstrated in FIG. 9A, FIG. 9C and FIG. 9E.

Phage clones from families 2 and 3 of Table 2 exhibited lower selectivity and were not examined further.

The three peptides identified were examined further for binding spores of close relatives of Bacillus anthracis, namely, Bacillus cereus and Bacillus thuringiensis. All three peptides demonstrated preferential binding to Bacillus anthracis. The phage carrying the peptide having the amino acid sequence DARGTTHM bound to Bacillus anthracis 3.7 fold better than to Bacillus cereus and 2.1 fold better than to Bacillus thuringiensis. Phage bearing the peptide having the amino acid sequence EPTRLSPHS bound to Bacillus anthracis 3.5 fold better than to Bacillus cereus and 6.9 fold better than to Bacillus thuringiensis. Phage bearing the peptide having the amino acid sequence ETRVPHGA bound to Bacillus anthracis 2.4 fold better than to Bacillus cereus and 2.2 fold better than to Bacillus thuringiensis. This is graphically depicted in FIG. 9B, FIG. 9D and FIG. 9F.

Accordingly, the analysis has identified three peptides that when incorporated into landscape phage clones bound to Bacillus anthracis spores at a higher rate than to other species of Bacillus spores. It is contemplated that landscape phage clones incorporating the peptides identified above would operate well as diagnostic probes for monitoring Bacillus anthracis spores by various platforms, as described above, in which antibodies or peptides have been previously used. Since the isolated landscape phage clones incorporating the peptides identified bind strongly to Bacillus anthracis spores, they may be used for separation and purification of spores before their identification by PCR, amino assays, cytometry, or other methods.

Additionally, based on common motifs, it is the inventor's conclusion that the following peptides will also selectivity and specifically bind Bacillus anthracis spores: AGRAGGGV, AGRGPGLP, ANRVPPTS, ATRPASSM, DARGTTHM, EPRLSPHS, ETRVPHGA, VDRGSATS, VDRGTTLS, VPRPDATS, VSDRGTAT, VSRIPSET, and VTRGSMNT. The analysis above is also identified isolated peptides that have selectivity in binding Bacillus anthracis spores. The peptides having selectivity in binding Bacillus anthracis spores are preferably selected from the following group: DARGTTHM, EPRLSPHS, ETRVPHGA, EPRAPASL, EPKPHTFS, EPHTKTST, DRTGATLT, EKTPVTAT, ERTVATTQ, VSQPASPS and VTRNTSAS. Aforementioned, the inventors contemplate incorporating all of the isolated peptides identified above into a diagnostic probe. A diagnostic probe is preferably a landscape page probe but may alternatively be an antibody probe or any other type of probe well known in the art.

### Materials and Methods

Strains and Spore Preparation

The Sterne strain of B. anthracis (an avirulent veterinary vaccine strain), Bacillus cereus T, and B. thuringiensis subsp. kurstaki were obtained from the US Army Medical Research Institute of Infectious Diseases (Fort Detrick, Md.). Bacillus subtilis (trpC2) 1A700 (originally designated 168) and B. licheniformis 5A36 (originally ATCC 14580) were provided by the Bacillus Genetic Stock Center, Ohio State University (Columbus, Ohio). B. megaterium ATCC 14581 was purchased from the American Type Culture Collection (ATCC)(Manassas, Va.). Spores were produced by cells grown in liquid Difco sporulation medium at 37° C. for 48-72 h with shaking. Remaining vegetative cells and cell debris were removed with a renografin step gradient. Spores were stored in sterile distilled water at 4° C.

Phage Library

The f8/8 landscape phage library, containing ˜2×10⁹ different clones, was described by the inventors and is detailed in an article entitled “A library of organic landscapes on a filamentous phage” in Protein Engineering, 2000; 13: 589-92, the subject matter of which is hereby incorporated by reference. The library was constructed by replacing amino acids E2, G3, and D4 on every copy of the pVIII coat protein of vector f8-1 (fd-tet derivative) with eight random amino acids.

Phage Growth, Purification, and Titering

The general procedures used for recombinant phage production and analysis, including media and buffers, are detailed in Phage Display: A Laboratory Manual, Cold Spring Harbor Laboratory Press, Cold Spring Harbor, N.Y., USA, 2001. Briefly, phage were propagated by infection of Escherichia coli K91 BlueKan cells, followed by growth of the infected cells for 16 h in NZY medium containing 20 mg/L tetracycline. Phage were purified by double polyethylene glycol precipitation. The total number of viral particles present in phage preparations was determined spectrophotometrically by use of the formula:

$$\frac{Virions}{mL} = \frac{{A}_{269} ⨯ ( 6 ⨯ {10}^{16} )}{number of nucleotides in the phage genome}$$

For the recombinant phage used in this work (9198 nucleotides), the formula:
Absorbance unit₂₆₉=6.5×10¹² virions/mL
was used to determine the concentration of phage particles in a solution (physical titer). The concentration of infective phage particles (biological titer) of a phage solution was determined by infection of starved K91 BlueKan cells with the phage, followed by their spreading on a tetracycline-containing agar plate. The recombinant phage carry the gene necessary for tetracycline resistance, allowing only those cells infected by phage to form colonies on the plate. The biological titer of these recombinant phage, expressed as colony-forming units (CFU) is typically 20-fold lower than the physical titer (virions/mL).
Selection of Spore-Binding Phage Clones

Bacillus anthracis Sterne spores (10⁷ in 25 μL of sterile distilled water) were applied to 8 wells of a Costar flat-bottom EIA/RIA 96-well plate. The plate was centrifuged for 2 min at 550 g to ensure an even coating of the wells with spores. The plate was then incubated at 37° C. overnight to dryness.

Wells containing Bacillus anthracis Sterne spores were blocked with 10 g/L bovine serum albumin (BSA) for 1 h at 37° C. The wells were then washed three times with 0.2 mL of Tris-buffered saline (TBS) containing 5 mL/L Tween 20 to remove unbound spores. The f8/8 phage library (1.25×10¹⁰ virions in 60 μL of TBS containing 5 mL/L Tween 20 and 0.1 g/L BSA) was added to each well and incubated 1 h at room temperature on an orbital shaker. Nonbound phage particles were then removed, and the wells were washed with 10 times with 0.2 mL of TBS containing 10 g/L BSA. Elution buffer (100 μL; 0.2 mol/L glycine-HCl, pH 2.2, containing 1 g/L BSA) was then added to each well and incubated for 5 min at room temperature. The eluates from all eight wells were transferred to a single microcentrifuge tube that was centrifuged for 3 min at 12 000 g and 4° C. to pellet any spores. The eluate was then neutralized by the addition of 140 μL of 1 mol/L Tris-HCl, pH 9.1, and concentrated by use of a Centricon 100 filter to a final volume of ˜100 μL. These concentrated phage clones were then propagated and purified for use in the next round of selection.

In the second round of selection, the phage clones that were selected and amplified in the first round were added to the spore-coated wells (rather than the primary phage library), but all other procedures remained the same. Likewise, in each subsequent round the phages selected and amplified in the previous round were added to the spore-coated wells. After the fourth round of selection, individual phage clones were amplified and sequenced to determine the amino acid sequences of the displayed peptides.

Phage-Capture Assay

Bacillus anthracis spores (2×10⁷ in sterile distilled water) were added to each well of a 96-well flat-bottomed microtiter plate. The plate was centrifuged for 2 min at 550 g and then incubated at 37° C. overnight to dryness. BSA (10 g/L) was added to the wells containing spores, and the plate was incubated for 1 h at 37° C. The wells were then gently washed with TBS containing 5 mL/L Tween 20. Candidate or control phages (˜10⁶ CFU in 50 μL of TBS) were added to separate spore-containing wells. After incubation for 1 h at room temperature, the plate was gently washed with TBS containing 5 mL/L Tween 20. Elution buffer (100 μL) was added to wells containing phages bound to immobilized spores and incubated for 5 min at room temperature. The eluates from each of these wells were transferred to sterile tubes and neutralized with 20 μL of 1 mol/L Tris-HCl, pH 9.1. Phage input and eluate were titered as described previously.

Biotinylation of Bacillus anthracis Sterne Spores

To biotinylate spores, we mixed 160.7 μL of 1.49 mmol/L Sulfo-NHS-LC-LC-Biotin (cat. no. 21338; Pierce, Biotechnology, Rockford, Ill., USA) in 2 mmol/L sodium acetate with 3.954×10⁹ spores in 1 mL of phosphate-buffered saline and incubated the mixture for 2 h at room temperature. Tris-HCl (300 μL; 1 mol/L, pH 9) was added, and the solution was incubated for 1 h to inactivate the remaining biotinylating agent. Spores were then centrifuged for 10 min at 9000 g and washed with water.

ELISA with Biotinylated Spores

Phage preparations (3×10¹⁰ virions in 60 μL) were loaded in a flat-bottomed 96-well microtiter plate and incubated at 4° C. for 12 h. The plate was washed with TBS containing 5 mL/L Tween 20 in a BIO-TEK ELₓ405 auto plate washer. Biotinylated Bacillus anthracis spores (5×10⁷ in 50 μL of TBS containing 5 mL/L Tween 20) were applied to the phage-coated wells and incubated for 2 h at room temperature on a rocker. The plate was then washed as before. Alkaline phosphatase conjugated to streptavidin (1 mg/L; Pierce) was added, and the plate was incubated for 1 h at room temperature on a rocker. After a final washing step, alkaline phosphatase substrate, p-nitrophenylphosphate, was added to the wells, and the absorbance at 405 nm (reference wavelength, 490 nm) was monitored for 1 h by an EL808 Ultra Microplate Reader (BIO-TEK Instruments, Inc., Winooski, Vt., USA).

ELISA with Nonbiotinylated Spores

The phage preparations (2.75×10¹⁰ virions in 55 μL of TBS) were loaded into each well of a flat-bottomed 96-well microtiter plate, and the plate was incubated overnight at 4° C. The plate was then washed as described above. Bacillus anthracis spores (2.5×10⁸ spores in 50 μL of TBS containing 5 mL/L Tween) were added to each well, and the plate was incubated for 2 h at room temperature with rocking. The plate was washed as before; 45 μL of the anti-Bacillus anthracis spore monoclonal antibody BD8 (2.2 mg/L) was added; and the plate was incubated for 1 h. The plate was washed again, and then 40 μL of goat anti-mouse IgG-alkaline phosphatase conjugate (22.9 μg/L; cat. no. S3721; Promega Corp., Madison, Wis., USA) was used for detection. The substratep-nitrophenylphosphate was then added, and the reaction was monitored as described above.

Coprecipitation Assay

Candidate phages (200 μL; 10⁶ CFU/mL) were mixed with 2×10⁷ Bacillus anthracis spores and/or other Bacillus species spores in a microcentrifuge tube and incubated for 1 h at room temperature on a rotator. Spore-phage complexes were pelleted by centrifugation for 10 min at 3000 g. The pellets were gently washed five times with 200 μL of TBS containing 5 mL/L Tween 20, and then suspended in 200 μL of elution buffer and incubated for 10 min at room temperature with occasional vortex-mixing. The spores were pelleted by centrifugation for 10 min at 9000 g, and the supernatant containing eluted phages was transferred to a fresh sterile tube and neutralized with 38 μL of 1 mol/L Tris-HCl, pH 9.1. Phage input and recovery were determined by biological titering as described previously.

Various alternatives and embodiments are contemplated as being within the scope of the following claims particularly pointing out and distinctly claiming the subject matter regarded as the invention.

What is claimed is:

1. An isolated peptide having selectivity and specificity in binding Bacillus anthracis spores, the isolated peptide having an amino acid sequence selected from the group consisting of: AGRAGGGV, AGRGPGLP, ANRVPPTS, ATRPASSM, DARGTTHM, EPRLSPHS, ETRVPHGA, VDRGSATS, VDRGTTLS, VPRPDATS, VSDRGTAT, VSRIPSET, and VTRGSMNT.

2. The isolated peptide of claim 1, wherein the isolated peptide is incorporated into a diagnostic probe.

3. The isolated peptide of claim 2, wherein the diagnostic probe is a landscape phage probe.

4. The isolated peptide of claim 2, wherein the diagnostic probe is an antibody probe.

5. The isolated peptide of claim 2, wherein the diagnostic probe is incorporated into a continuous monitoring system for detection of Bacillus anthracis spores.

6. An isolated peptide having selectivity in binding Bacillus anthracis spores, the isolated peptide having an amino acid sequence selected from the group consisting of: DARGTTHM, EPRLSPHS, ETRVPHGA, EPRAPASL, EPKPHTFS, EPHPKTST, DRTGATLT, EKTPVTAT, ERTVATTQ, VSQPASPS and VTRNTSAS.

7. The isolated peptide of claim 6, wherein the isolated peptide is incorporated into a diagnostic probe.

8. The isolated peptide of claim 7, wherein the diagnostic probe is a landscape phage probe.

9. The isolated peptide of claim 7, wherein the diagnostic probe is an antibody probe.

10. The isolated peptide of claim 7 wherein the diagnostic probe is incorporated into a continuous monitoring system for detection of Bacillus anthracis spores.

11. An isolated peptide having selectivity and specificity in binding Bacillus anthracis spores, the isolated peptide having an amino acid sequence selected from the group consisting of: DARGTTHM, EPRLSPHS, and ETRVPHGA

12. The isolated peptide of claim 11, wherein the isolated peptide is incorporated into a diagnostic probe.

13. The isolated peptide of claim 12, wherein the diagnostic probe is a landscape phage probe.

14. The isolated peptide of claim 12, wherein the diagnostic probe is an antibody probe.

15. The isolated peptide of claim 12, wherein the diagnostic probe is incorporated into a continuous monitoring system in detection of Bacillus anthracis spores.
